(19) United States
(12) Patent Application Publication
(10) Pub. No.: US 2021/0137041 A1
(43) Pub. Date: May 13, 2021

(54) PROCESSES FOR PRODUCTION OF LARGE QUANTITIES OF UNIFORM POTATO TUBERS FROM TRUE SEEDS

(71) Applicant: Agventure B.V., Wageningen (NL)

(72) Inventors: Michiel Erik DE VRIES, Holthees (NL); Julia Elisabeth STOCKEM, Dieden (NL); Willem Hendrik LINDHOUT, Wageningen (NL); Edwin VAN NIEUWENHUIZEN, Middenbeemster (NL); Menno TER MAAT, Wageningen (NL)

(21) Appl. No.: 17/091,702

(22) Filed: Nov. 6, 2020

Related U.S. Application Data
(60) Provisional application No. 62/933,151, filed on Nov. 8, 2019.

(51) Int. Cl. A01H 4/00; A01C 1/02; A01M 7/00

(52) U.S. Cl. CPC A01H 4/005; A01M 7/00; A01C 1/02

(57) ABSTRACT

Processes for the production of large quantities of diploid F1 Solanum potato tubers that are uniform in shape are provided. Diploid F1 hybrid plants are produced from true seeds. The tubers produced by the diploid F1 hybrid plants are either harvested for consumption, processing or extraction, or for production of seedling tubers which are then planted to produce tubers for consumption, processing or extraction. Also provided are diploid F1 hybrid seeds and plants that produce large quantities of tubers that are uniform in shape and produce large quantities of sprouts per tuber when used as seedling tubers.

### CROSS-REFERENCE TO RELATED APPLICATIONS

This application claims the priority of U.S. Provisional Appl. Ser. No. 62/933,151, filed Nov. 8, 2019, which is incorporated herein by reference in its entirety.

### FIELD OF INVENTION

This invention relates to processes for the production of large quantities of diploid F1 hybrid Solanum potato tubers that exhibit uniformity in shape, flesh color and skin color. More specifically, diploid F1 hybrid plants are produced from true seeds. The tubers produced by the diploid F1 hybrid plants are harvested for consumption. Alternatively, the tubers produced by the diploid F1 hybrid plants are replanted as seedling tubers for production of tubers for fresh consumption or food processing or extraction. This invention also relates to diploid F1 hybrid seeds and plants that produce large quantities of tubers that exhibit uniformity in shape of tubers from different individual plants, and germinate with ample sprouts per tuber when used as seedling tubers. Furthermore, this invention relates to the diploid, fertile, self-compatible and highly homozygous Solanum potato inbreds that produce F1 hybrid seeds upon crossing and the derived plants produce large quantities of tubers that exhibit uniformity in shape and germinate with ample sprouts per tuber when used as seedling tubers.

### BACKGROUND OF THE INVENTION

The tuber-bearing potato Solanum species is the third largest human food crop in the world after wheat and rice. In 2017, worldwide potato production was more than 388 million metric tons. China and India are the largest potato producers.

Unlike other major field crops, the vast majority of potatoes are commercially reproduced vegetatively (asexually) using potato tubers. The potato tubers used to produce new potato plants through vegetative reproduction are called “seed potato tubers” or “seed potatoes.” Seed potato tubers are not produced sexually through production of sexually reproduced seed. Seed potato tubers are produced through asexual or vegetative propagation of tubers. Because potato is reproduced vegetatively (asexually), a significant portion of each year's crop is set aside for planting of seed potato tubers in the next planting season. About 5% to 15% of each year's crop is saved for replanting next year's crop instead of being used to feed people.

In the developing world, most farmers select and store their own seed potato tubers for planting of next year's crop. It is very common that these seed potato tubers are diseased and are infected with viruses, bacteria, fungi and nematodes. In developed countries, farmers are more likely to buy disease-free “certified seed potato tubers” from dedicated suppliers. The certified seed potato tubers are aseptically produced in vitro and therefore are less likely to transmit viruses, bacteria, fungi and nematodes to progeny. The production of certified seed potato tubers though vegetative propagation is expensive and this same planting material is much too expensive for use in the developing world.

Seed potato tubers attract and transport pests and diseases. These pests and diseases include late blight, Andean potato weevils, nematodes, fungi, bacteria, tuber moths and viruses. Viruses are transmitted in the field by aphids and then transmitted through vegetative propagation to seed tuber potatoes. Virus infection of potato can decrease yields by as much as 20 percent. Potato seeds that are produced by sexual reproduction do not transmit viruses, bacteria or fungi and produce potato plants that are virus-free.

Another disadvantage of seed potato tubers is the high cost of transporting them. In developed countries, the disease-free seed potato tubers are frequently transported long distances to the place where seed potato tubers are planted to produce the potato crop. In addition to the cost of transport, planting of seed potato tubers have a significant carbon footprint.

Yet another disadvantage of current seed potato tuber production methods, as well as current breeding methods, is that they rely on use of tetraploid potato plants. Conventionally made diploid potato tubers are generally too small for commercial applications. In addition, the tetraploid genome is extremely heterozygous, often containing multiple alleles per locus. In a typical progeny plant produced from the cross of two unrelated tetraploid parent lines, deleterious alleles may contribute to reduced fitness in the case of homozygosity. Heterozygosity is likely to lead to increased vigor in tetraploid breeding. Tetraploid breeding reduces the likelihood the breeder can combine the better alleles among the 30,000 genes in one progeny plant and therefore it is very difficult to create beneficial combinations of agronomically desirable traits.

Because large populations of tetraploid progeny are needed to identify a potentially new potato selection with a new and attractive phenotype, the development of a new potato cultivar can often take up to eight years or more. A typical tetraploid breeding scheme produces 100 000 clones very year. In addition to the time required to breed the new cultivar, five or more years will be needed to asexually propagate sufficient quantities of seed potato tubers for food production. Despite the importance of potato as a food source, yield increase by genetic gain has been limited for the last century. The most important factor limiting genetic gain is the lack of an efficient breeding system. An advanced system is hybrid breeding based on crosses between two homozygous inbred lines. By choosing contrasting parents, yield contributing alleles can be combined. The absence of a self-compatibility system hampered the creation of homozygous inbred lines in potato. Furthermore, there was a need for methods to overcome inbreeding depression to produce a hybrid that is capable of producing sufficient seedling potatoes to produce a commercial crop for consumption.

Severe inbreeding depression and self-incompatibility in diploid potato germplasm have hitherto blocked the development of inbred potato lines. In some crops, such as leek and carrot, highly homozygous lines are not used commercially because their performance is too weak for hybrid seed production.

One of the objectives of the present invention is to use a diploid hybrid breeding system to increase the yield of potato tubers per hectare. Another objective of the present invention is to produce sufficient quantities of high quality seedling tubers from the cross of diploid, self-compatible, fertile and highly homozygous Solanum potato plants, that production of a commercial potato crop is feasible. It is another objective of the present invention to produce F1 diploid hybrids that produce large quantities of tubers per hectare when planted out in a field that are uniform in shape. It is another objective of the present invention to provide processes for the production of potato tubers in which seed of a diploid F1 hybrid Solanum species plant are germinated, potato plants are cultivated from said germinated seed which produce seedling tubers, the seedling tubers are collected and planted to produce a commercial potato tuber crop with a yield comparable to that of tetraploid varieties and tubers with a highly uniform shape.

### SUMMARY OF INVENTION

It is shown herein that production of commercial quantities of diploid, self-compatible hybrid Solanum potato tubers can be produced through propagation and planting of seedling potatoes. Based on these findings, methods and germplasm for production of commercial quantities of diploid, self-compatible hybrid Solanum potato tubers are provided.

The foregoing and other features of the disclosure will become more apparent from the following detailed description, which proceeds with reference to the accompanying figures.

Thus in one embodiment, there is provided a diploid F1 hybrid Solanum potato plant that produces at least 1 kilogram of tubers per square meter and the coefficient of variation in tuber shape is less than 26 percent.

In another embodiment of the invention, there is provided a diploid F1 hybrid Solanum potato plant that produces at least 5 kilograms of tubers per square meter.

In a further embodiment of the invention, there is provided a diploid F1 hybrid Solanum potato plant produces tubers having a coefficient of variation in tuber shape is less than 22 percent.

In yet another embodiment of the invention, there is provided a diploid F1 hybrid Solanum potato plant that produces at least 30 tubers per square meter and the coefficient of variation in tuber shape is less than 26 percent.

In a further embodiment of the invention, there is provided a diploid F1 hybrid Solanum potato plant that produces at least 60 tubers per square meter.

In a further embodiment of the invention, there is provided a the diploid F1 hybrid Solanum potato plant has a coefficient of variation in tuber shape of less than 22 percent.

In a further embodiment of the invention, there is provided a method of producing uniform potato tubers comprising the steps of (i) sowing Solanum potato seed individually in a receptacle such as a plug tray; (ii) growing said seed into seedlings for 3-8 weeks under protected growth conditions, at 15-25° C. and >30% humidity, while supplying light levels of at least 50 μMol/m²/sec during the light phase of a diurnal pattern after germination; (iii) mechanically transplanting the seedlings and growing the seedlings at a temperature of more than 0° C., while maintaining a matric potential pF at between 2 and 4.2 during at least the first three days while supplying light levels of at least 50 μMol/m²/sec during the light phase of a diurnal pattern; (iv) harvesting seedling tubers when the seedlings have reached a height of 60-120 cm; and (v) optionally, planting the seedling tubers in a subsequent crop cycle to produce uniform potato tubers.

Said uniform potato tubers are characterized by a uniform tuber shape, a uniform tuber number per plant, a uniform tuber weight per plant, or a combination thereof.

Said potato tubers are essentially homozygous, diploid Solanum potato tubers or essentially heterozygous, diploid Solanum potato tubers such as essentially heterozygous, diploid Solanum potato tubers are obtained by crossing essentially homozygous diploid parent lines.

In methods of the invention, the Solanum potato seed are sown individually, for example in a plug tray.

In methods of the invention, the seed are grown for 3-8 weeks in a greenhouse.

In methods of the invention, the seeds are sown with a spacing of at least 4 cm between the seeds.

In methods of the invention, the seedling tubers are planted with a spacing of at least 15 cm between the seedling tubers, such as a spacing of 20-50 cm.

In methods of the invention, seedlings are mechanically transplanted when the seedlings have more than 4 leaves, a height of more than 3 cm above soil surface, or both.

In methods of the invention, seedling tubers are planted that have a square measure of more than 25 mm and/or have no visual defects.

In methods of the invention, the seedling tubers that are planted have a sprouting score of at most 5.

In methods of the invention, the Solanum potato seed is grown at 15-20° C. and >80% humidity until germination, and at 15-25° C. and >30% humidity thereafter.

In methods of the invention, the Solanum potato seed was obtained by a method comprising the steps of (a) crossing a plant of a first diploid, self-compatible, fertile, highly homozygous potato line with a plant of a second diploid, self-compatible, fertile, highly homozygous potato line, whereby the second potato line differs in at least 20 homozygous loci as determined by molecular marker analysis, when compared to said first potato line; (b) collecting seeds produced from said cross.

In methods of the invention, the trait of self-compatibility is controlled by a SU gene.

In methods of the invention, the harvested seedling tubers were stored for at least one month. Said storage for at least one month preferably is performed at a temperature of 2-10° C.

In a further embodiment of the invention, there is provided a collection of essentially diploid potato tubers obtained from a single plant that, when planted, produce potato plants with a coefficient of variation in tuber shape of less than 26 percent. Said coefficient of variation in tuber shape preferably is less than 22 percent, such as less than 20 percent. Said essentially diploid potato tubers preferably are essentially diploid potato seedling tubers.

In a further embodiment of the invention, there is provided an essentially diploid potato plant with a coefficient of variation in tuber shape of less than 26 percent. Said coefficient of variation in tuber shape preferably is less than 22 percent, such as less than 20 percent. Said essentially diploid potato plant preferably is a diploid F1 hybrid Solanum potato plant. Said essentially diploid potato plant preferably produces at least 30 tubers per square meter.

In yet another embodiment of the invention there is provided a first diploid, highly homozygous, fertile and self-compatible Solanum potato inbred plant, wherein said inbred plant can be crossed with a second diploid, highly homozygous, fertile and self-compatible inbred plant to produce a diploid F1 hybrid Solanum potato plant that produces at least 1 kilogram of tubers per square meter and the coefficient of variation in tuber shape is less than 26 percent.

In a further embodiment of the invention there is provided a method of producing diploid and uniform Solanum potato tubers comprising the steps of (a) crossing a first diploid, self-compatible, fertile, highly homozygous Solanum potato inbred with a second diploid, self-compatible, fertile, highly homozygous Solanum potato inbred, (b) collecting seeds produced from said cross, (c) planting said seeds to produce seedling plants that produce at least 5 seedling tubers per square meter in the size range of 35-55 mm, (d) harvesting said seedling tubers, and (e) planting said seedling tubers to produce diploid and uniform potato tubers. The method may provide seedling plants that produce at least 6, 7, 8 or more than 8 seedling tubers per square meter in the size range of 35-55 mm. The seedling tubers may have a sprouting score of at least 5. The trait of self-compatibility may be controlled by the Sli gene. The diploid and uniform potato tubers have a coefficient of variation in tuber shape of less than 26 percent or less than 22 percent.

In a further embodiment of the invention there is provided a method of producing diploid and uniform Solanum potato tubers comprising the steps of (a) crossing a first diploid, self-compatible, fertile, highly homozygous Solanum potato inbred with a second diploid, self-compatible, fertile, highly homozygous Solanum potato inbred, (b) collecting seeds produced from said cross, (c) planting said seeds to produce seedling plants that produce seedling tubers, (d) harvesting said seedling tubers; and (e) planting said seedling tubers to produce a crop of commercial tubers wherein the yield of said commercial tubers is at least 1 kilogram per square meter and the coefficient of variation in tuber shape is less than 26 percent. The yield of commercial tubers may at least 5 kilograms per square meter. The coefficient of variation in tuber shape may be less than 22 percent. The seedling tubers may have a sprouting score of at least 5. The trait of self-compatibility may conferred by the Sli gene.

In yet another embodiment of the invention there is provided a method of producing diploid and uniform Solanum potato tubers comprising the steps of (a) crossing a first diploid, self-compatible, fertile, highly homozygous Solanum potato inbred with a second diploid, self-compatible, fertile, highly homozygous Solanum potato inbred, (b) collecting seeds produced from said cross, (c) planting said seeds to produce seedling plants that produce seedling tubers, (d) harvesting said seedling tubers; and (e) planting said seedling tubers to produce Solanum potato tubers wherein the yield of said tubers is at least 30 tubers per square meter and the coefficient of variation in tuber shape is less than 26 percent. The yield of commercial tubers may be at least 60 tubers per square meters. The coefficient of variation in tuber shape may be less than 22 percent. The seedling tubers may have a sprouting score of at least 5. The trait of self-compatibility may conferred by the Sli gene.

In a further embodiment of the invention, there is provided a method of producing diploid and uniform Solanum potato seedling tubers comprising the steps of (a) crossing a first diploid, self-compatible, fertile, highly homozygous Solanum potato inbred with a second diploid, self-compatible, fertile, highly homozygous Solanum potato inbred, (b) collecting seeds produced from said cross, (c) planting said seeds to produce seedling plants that produce at least 5 tubers per square meter in the size range of 28-55 mm. The seedling plants may produce at least 20 or at least 30 tubers per square meter in the size range of 28-55 mm. The trait of self-compatibility may be conferred by the SU gene.

In yet another embodiment of the invention, there is provided a method of producing diploid and uniform Solanum potato seedling tubers comprising planting seeds of a diploid F1 Solanum potato hybrid to produce seedling plants that produce at least 5 tubers per square meter in the size range of 28-55 mm. The plants may produce at least 20 or 30 commercial tubers per square meter in the size range of 28-55 mm.

### BRIEF DESCRIPTION OF THE DRAWINGS

The accompanying drawings, which are incorporated into and constitute a part of this specification, illustrate one or more examples of embodiments and, together with the description of example embodiments, serve to explain the principles and implementations of the embodiments.

FIG. 1 shows the frequencies in the number of seedling tubers (35-55 mm) produced per plant.

FIG. 2 shows a biplot of yield and PC1 of the AMMI analysis (Additive Main effects and Multiplicative Interaction Models).

FIG. 3 shows the average dry matter (DM) percentage and coefficient of variation (CV) of DM % of diploid hybrids and tetraploid cultivars.

FIG. 4 shows the average tuber shape and coefficient of variation (CV) of tuber shape of diploid hybrids and tetraploid cultivars.

FIG. 5 shows the boxplots of shape of individual tubers of tetraploid cultivar Bintje (n=3484) and diploid Hybrid 9 (n=2782) grown at five different locations in 2017.

FIG. 6 shows the contribution of more tubers per stem to total yield, expressed in yield increase per extra tuber/m2, compared to average number of tubers per stem over five sites (Berthem, Giethoorn, Hilvarenbeek, Est, Warmeriville). Cultivars that were used in the analysis are shown in blue. To verify that these cultivars are representative, all cultivars that were grown the yield trials are shown.

FIG. 7 shows the contribution of more stems per m2 to total yield, expressed in total yield increase per extra stem/m2, compared to average number of stems/m2 over five sites (Berthem, Giethoorn, Hilvarenbeek, Est, Warmeriville).

FIG. 8 shows the contribution of larger tubers to total yield, expressed in total yield increase per extra g tuber (tuber size), compared to average tuber size over five sites (Berthem, Giethoorn, Hilvarenbeek, Est, Warmeriville).

FIG. 9 shows the germination percentage (y-axis) of five different breeding lines at 14 days after sowing, on a temperature gradient table from 10-30° C.

FIG. 10 shows the germination percentage of a diploid hybrid on a temperature gradient table between 12 and 30° C.

FIG. 11 shows fresh weight production (mg) per seedling for 10 different hybrids, grown under 50, 100, 150 and 300 mol/m2/s.

FIG. 12 shows seedlings grown under 50, 100, 150 and 300 mol/m2/s (from left to right).

FIG. 13 shows the mean fresh weight of seedlings from two different hybrids grown under different EC levels of fertilizer.

FIG. 14 shows survival rates of seedlings of two different hybrids transplanted at four different time points.

FIG. 15 shows level of sprouting of seed tubers that were obtained from plantlets (seedlings) as starting material (A) or from tubers as starting material (B). Seed tubers were stored under three different conditions. A score of 9 meaning no sprouting and 1 meaning completely sprouting with long white sprouts.

### DEPOSIT INFORMATION

Seeds of Solynta lines AGVD1, AGVD2, AGVD3, and AGVD17 were deposited pursuant of the terms of the Budapest Treaty with the NCIMB, Ferguson Building, Craibstone Estate, Bucksburn, Aberdeen, AB21 9YA, Scotland, under NCIMB Accession Nos. 41663, 41664, 41665, and 41765, respectively. The date of deposit of lines AGVD1, AGVD2, and AGVD3 was Oct. 23, 2009 and the date of deposit of line AGVD17 was Oct. 5, 2010. Upon issue of a patent, all restrictions upon the deposits will be removed, and the deposits are intended to meet the requirements of 37 CFR section 1.801-1.809. The deposits will be irrevocably and without restriction or condition released to the public upon the issuance of a patent and for the enforceable life of the patent. The deposits will be maintained in the depository for a period of 30 years and will be replaced if necessary during the period.

### DETAILED DESCRIPTION OF THE INVENTION

The following explanations of terms and methods are provided to better describe the present disclosure and to guide those of ordinary skill in the art in the practice of the present disclosure. As used herein “comprising” means “including” and it is not intended to mean that the compositions and methods exclude elements that are not recited. “Consisting of” shall mean excluding more than a trace amount of other ingredients and substantial method steps recited. Embodiments defined by each of these transition terms are within the scope of this invention. The singular forms “a” or “an” or “the” include plural references unless the context clearly dictates otherwise. Thus, for example, a reference to “a compound” includes a plurality of compounds, and a reference to “a molecule” is a reference to one or more molecules. Similarly, reference to “comprising a therapeutic agent” includes one or a plurality of such therapeutic agents. The term “or” refers to a single element of stated alternative elements or a combination of two or more elements, unless the context clearly indicates otherwise. For example, the phrase “A or B” refers to A, B, or a combination of both A and B. Furthermore, the various elements, features and steps discussed herein, as well as other known equivalents for each such element, feature or step, can be mixed and matched by one of ordinary skill in this art to perform methods in accordance with principles described herein. Among the various elements, features, and steps some will be specifically included and others specifically excluded in particular examples. All numerical designations, e.g., pH, temperature, time, concentration, amounts, and molecular weight, including ranges, are approximations which are varied (+) or (−) by 10%, 1%, or 0.1%, as appropriate. It is to be understood, although not always explicitly stated, that all numerical designations may be preceded by the term “about.” It is also to be understood, although not always explicitly stated, that the reagents described herein are merely exemplary and that equivalents of such are known in the art.

Unless explained otherwise, all technical and scientific terms used herein have the same meaning as commonly understood to one of ordinary skill in the art to which this disclosure belongs. Although methods and materials similar or equivalent to those described herein can be used in the practice or testing of the present disclosure, suitable methods and materials are described below. The materials, methods, and examples are illustrative only and not intended to be limiting. All references cited herein are incorporated by reference in their entirety.

In some examples, the numbers expressing quantities of ingredients, properties such as molecular weight, reaction conditions, and so forth, used to describe and claim certain embodiments are to be understood as being modified in some instances by the term “about” or “approximately.” For example, “about” or “approximately” can indicate +/−20% variation of the value it describes. Accordingly, in some embodiments, the numerical parameters set forth herein are approximations that can vary depending upon the desired properties for a particular embodiment. Notwithstanding that the numerical ranges and parameters setting forth the broad scope of some examples are approximations, the numerical values set forth in the specific examples are reported as precisely as practicable. The recitation of ranges of values herein is merely intended to serve as a shorthand method of referring individually to each separate value falling within the range.

To facilitate review of the various embodiments of this disclosure, the following explanations of specific terms are provided:

The term “about” is used to indicate a variation in value by +/−10% of the value, or optionally +/−5% of the value, or in some embodiments, by +/−1% of the value.

The term “commercial crop” is used to indicate potato tubers produced by Solanum potato plants for consumption either fresh, processed in the form of chips, fries, dried, milled, among others. Alternatively it refers to a crop used for non-food purposes, such as animal feed, bio-plastics or food ingredients.

The term “seed potato” is used to indicate the asexually reproduced Solanum plant part that is used to plant a commercial crop of potato tubers.

The term “seedling tuber” is used to indicate the tuber or tubers produced by a potato plant seedling that results from germination of a sexually reproduced true seed. The seedling tuber can be used as the commercial tuber crop for consumption. Alternatively, the seedling tuber can be stored and replanted as the plant part that generates plants that produce the commercial crop of potatoes that are then consumed.

The term “potato” is used herein to refer to plant material that is essentially of Solanum tuberosum but may include introgression segments from other tuber-bearing Solanum species such as S. chacoense, S. phureja, S. andigena, S. stenotunum, S. tarijense, S. bertaulti, S. verrucosum, S. bulbocastanum S. etuberosum, S. tarijense S. paucijugum S. pinnatisectum S. sparsipilum, S. neorossii, S. schenckii, S. commersonii, S. albornozii, S. polyadenium, S. albicans, S. immite, S. andreanum, S. cardiophyllum, S. chomatophilum, S. microdontum, S. acaule, S. palustre, S. brevicaule, S. berthaultii, S. hjertingii, S. bukasovii, S. jamesii, and S. demissum.

The term “crossing” as used herein refers to the fertilization of female plants (or gametes) by male plants (or gametes). The term “gamete” refers to the haploid reproductive cell (egg or sperm) produced in plants by mitosis from a gametophyte and involved in sexual reproduction, during which two gametes of opposite sex fuse to form a diploid zygote. The term generally includes reference to a pollen (including the sperm cell) and an ovule (including the ovum). “Crossing” therefore generally refers to the fertilization of ovules of one individual with pollen from another individual, whereas “selfing” refers to the fertilization of the ovules of an individual with pollen from the same individual. Crossing is widely used in plant breeding and with a diploid results in a mix of genomic information between the two plants crossed in which one chromosome from the female and one chromosome from the male for each chromosome pair is combined. This will result in a new combination of genetically inherited traits. Usually the progeny from a crossing are designated as “F1”. If the F1 is not uniform, the progeny are heterogeneous genetically and phenotypically, it is usually designated as a “F1 population.” “Selfing” of a homozygous plant will usually result in a genetically identical plant sincere there is no genetic variation. “Selfing” of an F1 will result in an offspring that segregates for all traits that have heterozygotic loci in the F1. Such offspring are designate “F2” or a “F2 population.”

When referring to “crossing” in the context of achieving the introgression of a genomic region segment, the skilled person will understand that in order to achieve the introgression of only a part of the chromosome of one plant into the chromosome of another plant, it is required that random portions of genomes of both parental line will be recombined during the cross due to the occurrence of crossing-over events in the production of the gametes in the parent lines. Therefore, the genomes of both parents much be combined in a single cell by a cross, where after the production of gametes from said cell and their fusion in fertilization will result in an introgression event.

The term “intercrossable” as used herein refers to the ability to yield progeny plants after making crosses between parent plants.

As used herein, the terms “introgressing”, “introgress” and “introgressed” refer to both a natural and artificial process whereby individual genes or entire chromosomes are moved from one individual, species, variety or cultivar into the genome of another individual, species, variety or cultivar by crossing those individuals, species, varieties or cultivars. In plant breeding the process usually involves selfing or backcrossing to the recurrent parent to provide for an increasingly homozygous plant having essentially the characteristics of the recurrent parent in addition to the introgressed gene or trait.

The term “introgression” refers to the result of an introgression event.

The term “backcross” refers to the result of a “backcrossing” process wherein the plant resulting from a cross between two parental lines is repeatedly crossed with one of its parental lines, wherein the parental line used in the backcross is referred to as the recurrent parent. Repeated backcrossing results in replacement of genome fragments of the donor parent with those of the recurrent parent. The offspring of a backcross is designated “BCx” or “BCx population” where “x” represent the number of backcrosses.

The term “backcrossing” as used herein refers to the repeated crossing of a hybrid progeny back to the recurrent parents. The parental potato plant which contributes the gene for the desired characteristic is term the nonrecurrent or donor parent. This terminology refers to the fact that the nonrecurrent parent is used one time in the backcross protocol and therefore does not recur. The parental potato plant to which the gene or genes from the nonrecurrent parent are transferred is known as the recurrent parent as it is used for several rounds in the backcrossing protocol. In a typical backcross protocol, the original variety of interest (recurrent parent) is crossed to a second variety (non=recurrent parent) that carries the single gene of interest to be transferred. The resulting progeny from this cross are then crossed again to the recurrent parent and the process is repeated until a potato plant is obtained where essentially all of the desired morphologic and physiological characteristics of the recurrent parent are recovered in the converted plant, in addition to the single gene or a limited number of genes transferred from the nonrecurrent parent.

The selection of a suitable parent is an important step for a successful backcrossing procedure. The goal of a backcross protocol is to later or substitute a single trait or characteristic in the original variety. To accomplish this, a single gene of the recurrent variety is modified, substituted or supplemented with the desired gene from the nonrecurrent parent, while retaining essentially all of the rest of the desired genes, and therefore the desired physiological and morphological constitution of the original variety. The choice of the particular nonrecurrent parent will depend on the purpose of the backcross. One of the major purposes is to add some commercially desirable, agronomically important traits to the recurrent parent. The exact backcrossing protocol will depend on the characteristic or trait being altered or added to determine an appropriate testing protocol. Although backcrossing methods are simplified when the characteristic being transferred is a dominant allele, a recessive allele may also be transferred. In this instance, it may be necessary to introduce a test of the progeny to determine if the desired characteristic has been successfully transferred. Preferably such genes are monitored by diagnostic markets.

Transgenes can be introduced into the plant using any of a variety of established recombinant methods well known to persons skilled in the art.

Many single gene traits have been identified that are not regularly selected for in the development of a new variety but that can be improved by backcrossing and genetic engineering techniques. Single gene traits may or may not be transgenic. Examples of these traits include but are not limited to herbicide tolerance, tolerance to bacterial, fundal or viral disease, tolerance to insect infestation, uniformity or increase in concentration of starch and other carbohydrates, enhanced nutritional quantity, decrease in tendency of the tuber to bruise, and decrease in the rate of starch conversion to sugars.

The term “selfing” refers to the process of self-fertilization wherein an individual is pollinated or fertilized with its own pollen. Repeated selfing eventually results in homozygous offspring.

A “line”, as used herein, refers to a population of plants derived from a single cross, backcross or selfing. The individual offspring plants are not necessarily identical to one another. It is possible that individual offspring plants are not vigorous, fertile, or self-compatible due to natural variability. However, it is foreseen that suitable plants that are vigorous, fertile and self-compatible can be easily identified in a line and used for additional breeding purposes.

As used herein the term “allele(s)” means any of one or more alternative forms of a gene, all of which alleles relate to at least one trait or characteristic. In a diploid cell or organism, the two copies of a gene occupy corresponding loci on a pair of homologous chromosomes. Each copy may be a distinct allele.

A “gene” is defined herein as a hereditary unit that occupies a specific location on a chromosome and that contains the genetic instruction for a contribution to potential phenotypic characteristics or trait in a plant. A gene may be defined by its nucleotide sequence.

A “locus” is defined herein as the position that a given gene occupies on a chromosome of a given species.

As used herein, the term “homozygous” means a genetic condition existing when identical alleles reside at corresponding loci on homologous chromosomes.

The terms “essentially homozygous” or “highly homozygous” refer to a level of homozygosity of at least 25%, preferably at least 50%, more preferably at least 75%, still more preferably at least 76, 77, 78, 79, 80, 81, 82, 83, 84, 85, 85, 86, 87, 88, 89, 90, 91, 92, 93, 94, 95, 96, 97, 98, 99, or 100% homozygosity when testing 100, preferably 1000, more preferably at least 10,000 loci. The skilled person will appreciate that the level of homozygosity of a plant is by definition the level of homozygosity as displayed across the whole genome of the plant, and that such testing of 100, preferably 1000, more preferably at least 10,000 loci reflects the level of homozygosity across the plant's genome, such for instance obtained by random selection of loci but this may depend on the markers use. Homozygosity levels are average values for the population and refer preferably to those loci wherein the parents differ.

As used herein, the “heterozygous” means a genetic condition existing when different alleles reside at corresponding loci on homologous chromosomes.

The term “recombination” or recombine” refers to the exchange of information between two homologous chromosomes during meiosis. In a “recombinant” plant, DNA that is originally present on a specific location within the chromosome, e.g. linked to a gene/locus, is exchanged for DNA from another plant (i.e. maternal for paternal or vice versa). In order to exchange only the required material, and maintain the valuable original information on the chromosome as much as possible, two flanking crossover or recombination events will usually be required. In a double recombinant this exchange has taken place on both sides of a gene/locus. One way to find such a double recombinant is to screen a population of F2 plants. This population has to be large since double recombination occurs in only a limited frequency. Alternatively, double recombinants within a genetic unit can be the result of subsequent backcrossing. The frequency of double recombination is the product of the frequencies of the single recombinants. A recombinant in a 10 cM area can be found with a frequency of 10% while a double recombinant is found with a frequency of 1%.

As used herein the term “progeny” means genetic descendants or offspring.

As used herein the term “population” means a genetically heterogeneous collection of plants sharing a common genetic derivation.

A “recombination event” refers to a mitotic or meiotic crossing-over event.

As used herein, the term “hybrid” means any offspring of a cross between two genetically unlike individuals. More preferably, the term hybrid refers to the cross between two elite or inbred breeding lines which will not reproduce true to the parent from seed.

The term “segregate” as used herein refers to the separation of paired alleles during meiosis so that members of each pair of alleles appear in different gametes. The term includes reference to the result of this genetic phenomenon wherein the offspring population of a crossing in which at least one of the parents is heterozygous for an allelic gene is non-uniform with respect to phenotypic that conferred by said gene.

The term “breeding line” as used herein refers to a line of a cultivated potato having commercially valuable or agronomically desirable characteristics, as opposed to wild varieties or land races. The term includes reference to an elite breeding line or elite line, which represents and essentially homozygous, usually inbred, line of plants used to produce commercial F1 hybrids. An elite breeding line is obtained by breeding and selection for superior agronomic performance comprising a multitude of agronomically desirable traits. An elite plant is any plant from an elite line. Superior agronomic performance refers to a desired combination of agronomically desirable traits wherein it is desirable that the majority, preferably all of the agronomically desirable traits are improved in the elite breeding line as compared to a non-elite breeding line. Elite breeding lines are essentially homozygous and are preferably inbred lines.

The term “elite line” as used herein refers to any line that has resulted from breeding and selection for superior agronomic performance. An elite line preferably is a line that has multiple, preferably at least 3, 4, 5, 6 or more desirable agronomic traits.

The terms “cultivar” and “variety” are used interchangeably herein and denote a plant which has deliberately been developed by breeding, e.g. crossing and selection, for the purpose of being commercialized by farmers or growers to produce agricultural products for consumption or processing, feed, etc. the term “breeding germplasm” denotes a plant having a biological status other than a “wild” status, which “wild” status indicates the original non-cultivated or natural state of a plant or accession.

The term “breeding germplasm” includes, but is not limited to, semi-natural, semi-wild, weedy, traditional cultivar, landrace, breeding material, research material, breeder's line, synthetic population, hybrid, founder stock/base population, inbred line (parent of hybrid cultivar), segregating population, mutant/genetic stock, market class and advanced/improved cultivar. Examples of cultivars include such cultivated varieties as Bintje, Russet Burbank, Eigenheimer, and Nicola.

As used herein, the terms “purebred, “pure inbred” or “inbred” are interchangeable and refer to a substantially homozygous plant or plant line obtained by repeated selfing and/or backcrossing.

As used herein, the term “molecular genetic marker” or “marker” refers to an indicator that is used in methods for visualizing differences in characteristics of nuclei culture acid sequences. Examples of such indicators are restriction fragment length polymorphism (RFLP) markers, amplified fragment length polymorphism (AFLP) markers, single nucleotide polymorphism s (SNPs), insertion/deletion (INDEL) mutations, microsatellite markers (SSRs), sequence-characterized amplified regions (SCARs), cleaved amplified polymorphic sequence (CAPS) markers or isozyme markers or combinations of the markers described herein which defines a specific genetic and chromosomal location.

As used herein the term “plant part” indicates a part of the potato plant, including tubers, portions or cuttings of tubers, organelles, single cells and cell tissues, pollen, ovules, berries, seeds, anthers, flowers stems, shoots and the like.

Unless expressly stated otherwise, the term “seed” as used throughout this specification, refers to the body from which a new plant develops containing the small embryonic plant enclosed in a seed coat covering, usually together with some stroe food. This seed, referred to as a botanical or “true” seed is the product of the ripened ovule of angiosperm or gymnosperm plants which occurs after fertilization and some growth within the mother plant.

As used herein, the terms “vigor” or “vigorous” refer to the relative amount of above-found or below ground tissue of plant, and/or the amount of seed produced by the plant, which relative amount may be more or less independent of one another. With regard to tubers, among the methods of evaluating vigor is the number of sprouts per seeding tuber.

Tuber yield is based on either tubers from a seedling plant, referred to herein as “seedling tubers”, or tubers form from tuber grown plants. The tuber yield can be expressed in a number of different ways including number of tubers per plants; tuber size; total tuber number of tubers per acre or hectare; total tuber fresh or dry weight per plant, or acre ‘or hectare; among others.

The term “a line comprising plants” is used herein to described plant having a certain characteristic, such as uniform tuber shape, but should preferably be understood as referring to a line essentially consisting of plant having the said trait while allowing some biological variation.

The term “diploid” as used here refers to a plant wherein each vegetative cell contains two sets of chromosomes (2x=2n).

The term “tetraploid” as used herein refers to a plant wherein each vegetative cell contains four sets of chromosomes (2x=4n).

The term “tuber skin color” as used herein refers to the color of the kin of a tuber after harvest as the result of anthocyanin accumulation in tuber skin tissues.

The term “tuber flesh color” as used herein refers to the color of the interior of the tuber flesh after harvest as the result of the absence or presence of carotenoid compounds causing white or yellow flesh color, respectively, as well as the absence of anthocyanin compounds causing red, blue, purple shades of flesh color, irrespective of the presence of color in patterns being partial for full.

The term “tuber starch content” as used herein refers to the starch weight over the total fresh weight of a tuber.

The term “tuber dormancy period” as used herein refers to the period after harvest when the conditions for sprouting are optimal and the tubers are not sprouting.

As used herein the term “tuber dry matter content” refers to the weight of the dry components of a potato tuber divided by the total fresh tuber weight.

The term “tuber shape” as used herein refers to the length/width ratio, to indicate the continuous variation from round, oval to long shapes, as well as the height/width ratio to indicate the continuous variation from cylindrical to the amount of flatness of a tuber.

The term “tuber uniformity” as used herein refers to the standard deviation of tuber traits, whereby by a low standard deviation refers to high uniformity and vice versa. Trait uniformity can be expressed as the coefficient of variation (CV) which is a measure of relative variability. CV is the ratio of the standard deviation to the mean (average). The higher the CV, the greater the level of dispersion around the mean. CV is generally expressed as a percentage.

The term “fertile offspring” or “fertile seed” is defined herein as seed capable of growing into a flower-producing potato plant wherein flowers are male and female fertile. Thus, the term preferably refers to a plant or seed that when grown into a plant is capable of producing offspring as male and as female parent by virtue of the presence of fertile ovules and fertile pollen (i.e. both male and female flowers are fertile).

The term “fertile plant” is defined herein as a plant capable of producing fertile seed carrying berries.

The term “self-compatible” refers to the capacity to develop seeds in berries that are the result of self-pollination, self-fertilization, and producing fertile progeny.

The term “germination” is defined herein as the moment of radicle protrusion from a swollen and imbibed potato seed.

The term “sprouting” is defined herein as the moment of breaking of dormancy, when the eyes of a potato tuber start to swell. The sprouting scoring method of Tiemens-Hulscher et al., 2013 (Tiemens-Hulscher et al., 2013. Potato Breeding. Drukkerij De Swart, Den Haag) was used.

The term “matric potential pF”, or “capillary potential” is defined herein as the energy with which water is held by the soil. The pF function is defined as the numerical value of the negative pressure of the soil moisture expressed in cm of water.

The term “square measure” is a traditional industry tool for classifying potato tubers according to square mesh size. Potato sizing squares from 30 to 80 mm, in steps of 5 mm, are used to assess the size and shape of potatoes, important for both yield and quality.

For other terms as used herein, reference is made to Allard, R. W., Principles of Plant Breeding, 2nd Edition, Wiley, New York (1999), and especially the glossary in this textbook.

The present invention provides methods of producing uniform potato tubers, especially when starting with true potato seed. As most modern potato varieties are tetraploid, their genetics is complex and most potatoes are vegetatively propagated.

An advanced system was recently developed that is termed hybrid breeding and which is based on crosses between two diploid, homozygous inbred lines (Lindhout et al., 2011. Potato Research 54: 301-312). By choosing contrasting parents, yield contributing alleles can be combined (Lindhout et al., 2011.). The segregation of potato traits after several generations of inbreeding was described by Meijer et al., 2018 (Meijer et al., 2018. Euphytica 214:121). Until now, the absence of a self-compatibility system hampered the creation of homozygous inbred lines in potato. Since the discovery of the Sli-gene (Hosaka and Hanneman, 1998. Euphytica 103: 265-271), it has become possible to develop a diploid hybrid breeding system (Lindhout et al., 2018. Achieving Sustainable Cultivation of Potatoes 1:1-24). The discovery not only creates opportunities for commercial breeding programs, it also greatly facilitates genetic research by using advanced segregating populations such as introgression libraries and backcross populations (Jeuken and Lindhout, 2004. Theor Appl Genet 109:394-401; Endelman and Jansky, 2016. Theor Appl Genet 129:935-943; Prinzenberg et al., 2018. Physiol Plant 164:163-175). Diploid germplasm allows high throughput phenotyping and efficient QTL detection, accelerating the breeding process (Meijer et al. 2018; Prinzenberg et al. 2018; Su et al., 2019. Am J Potato Res 97:33-42).

With the development of this hybrid breeding system, which is new for potato, using diploid hybrids instead of tetraploid clones, a system of testing new cultivars needs to be developed. Commonly in breeding systems, new cultivars are grown at several locations from tubers and their performance is benchmarked against a set of already existing commercial tetraploid cultivars (Tiemens-Hulscher et al., 2013. Potato Breeding. Drukkerij De Swart, Den Haag). To determine relevant genetic gain, performance of diploid hybrids needs to be benchmarked against tetraploid cultivars commonly used by farmers. Assessing the genotype by environment interactions in farmers' fields is an important aspect of acceptance of new (types of) cultivars.

The present invention provides diploid hybrid Solanum potato hybrids with yields and tuber shape uniformity equal to that of tetraploid commercial cultivars. Furthermore, the present invention provides the potato germplasm and methods necessary for breeding high yield and tuber shape uniformity into diverse diploid genetic backgrounds. The present invention demonstrates diploid Solanum potato hybrids have a yield potential comparable to commercial tetraploid cultivars. For major economically important traits such as dry matter percentage, tuber number/m2 and tuber size, the diploid hybrids of the present invention demonstrated equivalent performance to commercial tetraploid cultivars.

With regard to tuber yield, the best diploid Solanum potato hybrids performed as well as the lower yielding commercial tetraploid cultivars. Remarkably, the diploid Solanum potato hybrid cultivars of the present invention were never selected for tuber yield or tuber uniformity under field conditions. The Solanum potato hybrids of the present invention, and the diploid, fertile, highly homozygous inbreds used to make them through hybrid breeding, were grown and selected in greenhouses based largely on plant vigor and total seed production. Stability of traits such as tuber yield and tuber shape uniformity in the potato hybrids of the present invention was remarkably similar to that of commercial tetraploid cultivars across different geographic locations.

The present invention demonstrates that the components of yield, including sterns per square meter, tubers per stern and tuber size, impacted both diploid hybrid and tetraploid commercial cultivars in a similar fashion. In both commercial tetraploid cultivars and diploid hybrids, the number of tubers per stern had the greatest positive impact on total tuber yield. The number of sterns per meter squared had the next largest positive impact on total tuber yield. Tuber size had the smallest impact on total tuber yield.

It is likely that the tetraploid and outcrossing nature of commercial potato is responsible for the large genetic variation observed in this crop. In a study on the allelic composition of 800 genes in 83 potato cultivars, an average of 3.2 alleles per location within a genotype was identified. See, Uitdewilligen et al., PLoS ONE 8(5), e62355 (2013). Among the 83 cultivars, it was not uncommon to observe more than 10 alleles per locus. The high frequency of allelic variation has the inevitable consequence that “weak alleles” that have a negative effect on plant fitness are maintained. These alleles are revealed upon inbreeding, especially at the diploid level, where homozygosity increases much more rapidly upon inbreeding. However, this large genetic variation is very helpful for breeding as it forms a genetic reservoir of useful genes.

The potato germplasm available for breeding comprises many species, including diploid species. Another important source of diploid germplasm is tetraploids that can be prickle pollinated to generate diploid offspring designated dihaploids. The process of prickle pollination to generate diploid offspring is described in Uigtewaal et al. Theor. Appl. Genet. 73: 751-758 (1987), which is incorporated by reference in its entirety herein. A collection of dihaploids obtained from one tetraploid contains the full set of genes from the tetraploid and can be exploited in a diploid potato breeding program.

Sources of self-compatibility are available in Solanum germplasm. For example, the dominant self-compatibility controller gene Sli was described in S. chaconense by Hosaka, K. and Hanneman, R. E. Euphytica 99:191-197 (1998) and Hosaka, K. and Hanneman, R. E. Euphytica 103: 265-271 (1998). The Sli gene can be accessed by making crosses with any of Solynta lines AGVD1, AGVD2, AGVD3, or AGVD17, seeds of which were deposited with the NCIMB, Ferguson Building, Craibstone Estate, Bucksburn, Aberdeen, AB21 9YA, Scotland, under NCIMB Accession Nos. 41663, 41664, 41665, and 41765, respectively. From the deposited germplasm, inbreds that are each diploid, fertile, self-compatible, and highly homozygous can be produced. Intercrossing these inbreds produces hybrids with high tuber yield and tubers with a uniform shape. Alternatively, the self-compatibility trait can be accessed through knocking out the self-incompatibility gene S-RNase using CRISPR-Cas 9. See, for example, Mingwang et al., Nature Plants 4(9):651-654 (2018).

AGVD1 represents an F2 obtained by crossing as a first parent potato line designated IVP07-1001/4 with a second parent potato line designated D1. IVP07-1001/4 is essentially homozygous, diploid, fertile and self-compatible but not vigorous. IVP07-1001/4 is an essentially homozygous potato line obtained by multiple generations of selfing plants of a line derived from public germplasms which plants have the self-compatibility controlling SU gene of Phumichai et al., Euphytica 148: 227-234 (2006), which article is incorporated by reference herein. The IVP07-1001/4 plants grew poorly, had very few flowers, and each berry contained only a few seeds. The plants were very small and produced few tubers and those that did form were very small with an average tuber weight of 5 grams per tuber. D1 is an elite diploid breeding clone that is essentially non-homozygous and is not self-compatible but has the desirable agronomic trait of yellow flesh color and exhibits good cooking quality in that the flesh does not grey after cooling. The resulting F1 offspring plants from the cross of IVP07-1001/4 and D1 were selfed. It is estimated that AGVD1 is about 50% homozygous, meaning that about 50% of the loci for which the parents differ are homozygous.

AGVD2 represents a pseudo F2 (F1xF1) obtained by crossing IVP07-1001/4 with a plant of the parent D1 as indicated above, and mutually crossing individual F1 offspring plants that share the IVP07-1001/4 parent but have different individuals of the D1 parent. It is estimated that AGVD2 is about 25% homozygous for the loci for which the parents differ.

AGVD3 represents a BC1 population obtained by crossing a plant of parent IVP07-1001/4 with a plant of parent D1 and backcrossing the resulting F1 offspring plants with the IVP07-1001/4 parent. It is estimated that AGVD3 is about 75% homozygous for the loci for which the parents differ.

AGVD17 represents a F3 population obtained by crossing a plant of parent IVP07-1001/4 with parent D2. D2 is an elite diploid breeding clone that is essentially non-homozygous and is not self-compatible. D2 has the desirable agronomic traits of oval tubers, yellow flesh, and good chip production. The resulting F1 progeny were backcrossed with plants of IVP07-1001/4 followed by 2 steps of selfing. It is estimated that AGVD17 is greater than 80% homozygous for the loci for which the parents differ.

The deposited lines AGVD1, AGVD2, AGVD3, and AGVD17 were used to produce the inbreds and hybrids of the present invention which produce the tuber yields and uniform tuber shape described and claimed herein. It was unexpected that the inbreeding depression could be overcome during the process of selfing within this population of plants. Furthermore, it was unexpected that the right combination of genes could be assembled in inbreds to produce hybrids with the claimed levels of tuber production and uniformity in tuber shape. Using the present disclosure, a person skilled in the art can do multiple rounds of crossing by hand and self-pollination to arrive at the inbreds and hybrids described and claimed herein. What is particularly surprising is that the claimed inbreds were not selected for tuber yield or uniform tuber shape. The inbreds were selected in the greenhouse for vigor and seed production. Yet test crosses among the inbreds unexpectedly resulted in the identification of hybrids with excellent tuber yield and uniform tuber shape. By multiple rounds of selfing and crossing, deleterious alleles are removed from the population and homozygosity is increased each generation. The presented data show the results of hybrids produced by intercrossing inbred lines that were developed after more than 10 successive events of selfing and intercrossing.

The two main methods applied here are making crosses between two individual plants, cross-pollination and making a cross of a plant with itself, a so-called self-pollination. In the case of cross-pollination, the plant is grown from seed. Of the male plant, pollen is harvested from the male plant by gently tapping mature open flowers to release pollen and capturing released pollen in Eppendorf tubes. When the female plant enters generative stage, buds are selected for crossing. When the buds have a width of at least 2 mm, they are cut open to emasculate the buds to prevent self-pollination. Subsequently, pollen harvested from the male plant of the cross is applied on the stamen. Application is done by using a small metal nail, dipping it into the Eppendorf tube and touching with the pollen-filled nail the stamen of the female plant. The fertilized flower will develop into a berry and the seeds in that berry constitute the next generation of seeds. Seeds are removed from the mature berry by crushing the flesh and rinsing the flesh and seeds in water. The seeds will release from the flesh and form a supernatant. The seeds are then dried for storage. Seedling tubers can be produced in the greenhouse by sowing seeds in a medium with sufficient water and nutrient supply and at temperature between 15 to 20° C. and at least 12 hours of daylight per day. As soon as seedlings reach 5 to 10 cm in length, they are transplanted into pots. The seedlings can be phenotypically selected for all relevant traits, for example Phytophthora resistance, tuber yield, and tuber shape.

In methods of the invention, Solanum potato seed is sown in a receptacle. Said receptacle comprises a suitable soil for sowing, for example potting soil. Potting soil is a soil that won't compact around a plant's roots and drains well. Suitable potting soil comprises, for example, one or more of sphagnum peat moss, perlite, lime or limestone, vermiculite, compost, fertilizer, sand, and soil. Said receptacle preferably is a tray in which the seeds are sown at fixed position, for example in individual plugs. Said seeds are preferably sown with a spacing of at least 2 cm between the seeds, more preferred at least 2.5 cm between the seeds, more preferred at least 3 cm between the seeds, more preferred at least 3.5 cm between the seeds, more preferred at least 4 cm between the seeds, more preferred at least 4.5 cm between the seeds, more preferred at least 5 cm between the seeds. Although there is no maximal spacing between the seeds, for efficiency, the spacing is preferably less than 20 cm, more preferred less than 10 cm.

Germinating seeds need a soil that retains moisture, which is an essential component for germination. For this, the receptacle comprising the soil may be wetted with clean water until it is evenly moist. Seeds, preferably dried seeds, may be gently placed in the middle of every plug, for example by hand, where after the receptacle may be covered to avoid drying out, for example with a layer of vermiculite. After sowing the seed, the receptacle may be sprayed again with clean water to prevent drying.

The sown seed may be grown into seedlings for a number of weeks, for example 3-8 weeks, such as 4-7 weeks, 5-6 weeks, under protected growth conditions. It is preferred that the sown seed is sheltered against direct sunlight. Said protected growth conditions include, for example, a greenhouse, a glass/screen house, or a wooden shelter with thatched roof where the sown seeds can be placed under a transparent plastic tunnel to ensure a constant humid (RV>80%) environment. The seed is preferably grown at a moderate temperature, preferably at 15-25° C., such 16-24° C., 17-23° C., 18-22° C., and including 19° C., 20° C., and 21° C. It was found that elevated temperatures, especially a temperature above 25° C., hampered germination. The seed is preferably grown at a humidity of >30% humidity, such as >40% humidity, >50% humidity, >60% humidity up to 100% humidity.

After germination of the seed, or at least after a radicle becomes visible in or above the soil, the seedlings preferably are kept in a day-night light-dark cycle, with light levels of at least 50 μMol/m2/sec during the light phase of such diurnal pattern. Said light phase preferably comprises 6-24 hours, more preferred 8-16 hours, such as about 10-14 hours of light of at least 50 μMol/m2/sec. Said light levels preferably are 100-500 μMol/m2/sec, such as 200-400 μMol/m2/sec, including about 300 μMol/m2/sec. With temperatures between 15 and 22° C., the majority of seeds may be germinated in about 8 days.

After sowing, the seed may be grown into seedlings at 15-20° C. and >80% humidity until germination, and at 15-25° C. and >30% humidity thereafter. The soil of the seedlings needs to be kept moist. Seedlings may receive a small amount of water soluble fertilizer when the roots of the seedlings have reached the bottom of the receptacle. Said fertilizer preferably comprises 1-5 dS/m, more preferred 1-3 dS/m, which increases growth parameters like leaf area and biomass production, when compared to standard fertilization of approximately 0.9 dS/m.

Seedlings may be transplanted after a number of weeks. Transplantation preferably is performed when seedlings, on average, have at least 4 true leaves, more preferred when they have more than 4 leaves such as 5 leaves or 6 leaves. Transplantation preferably is performed when seedlings, on average, have reached a height of at least 3 cm above soil surface, such 4 cm above soil surface. Transplantation preferably is performed when seedlings, on average, both have at least 4 leaves and have reached a height of at least 3 cm above soil surface.

Transplantation may be performed by positioning the roots of the seedlings gently into a soil. In order not to damage the roots of the seedlings, it is preferred that the content of a receptacle in which the seeds were sown, substantially the complete content, is transplanted, including soil and roots. The whole clod preferably is covered with soil after transplantation. Seedlings are preferably positioned in ridges with a distance of 30-120 cm, such as 60-90 cm, between the ridges. Seedlings are preferably positioned at a depth of 1-5 cm, meaning that at least one cm of the stem of the seedlings is present above the soil. It is no problem to also put some of the leaves under the ground. From the lateral buds, new roots will develop. This will increase the root system of the seedling. About 70-80% of the total length of the seedling is to be covered with soil. Transplantation preferably is performed with a distance of 10-50 cm between the transplanted seedlings, preferably about 20-40 cm between two plants such as about 30-35 cm.

Said seedling may be transplanted in the field, or under protected growth conditions, at a temperature of at least 0° C., such as 1-35° C., 5-25° C., or 10-20° C. Prior to transplantation to the field, seedlings may be placed outside in the shadow for hardening for 3-4 days. Transplantation may be performed manually or mechanically, preferably mechanically. Immediately after transplanting the plants are to be irrigated, in order to maintain a matric potential pF at between 2 and 4.2 during at least the first three days after transplanting. If not, the transplant can dry out and become irreversible damaged. When it is raining, the rainfall must exceed 10 mm, otherwise the seedlings must be irrigated. When irrigated on time, it will ensure that the seedling makes good contact with the soil. The water will settle the seedling firm on its place. This way it can start growing right away. Light preferably is provided at levels of at least 50 μMol/m2/sec during the light phase of a diurnal pattern. Said light levels preferably are 100-500 μMol/m2/sec, such as 200-400 μMol/m2/sec, including about 300 μMol/m2/sec. A basic fertilizer may be used at regular intervals. Said basis fertilizer may comprise, for example nitrogen:phosphorus:potassium in a 5:1.6:3 (w/w) ratio. Said fertilizer preferably comprises 1-5 dS/m, more preferred 1-3 dS/m, which increases growth parameters like leaf area and biomass production, when compared to standard fertilization of approximately 0.9 dS/m. Herbs are preferably removed mechanically. To control late blight (Phytophthora infestans), seedlings may be contacted with fungicides such as metalaxyl, a carbamate compound, mandipropamid, chlorothalonil, fluazinam, triphenyltin, mancozeb, or a combination thereof.

Prior to harvesting, haulm of tuber seedlings may be removed, for example by mechanical-chemical killing, such as by crushing the foliage to 20-30 cm height, adding diquat dibromide (for example Reglone®; Syngenta Crop Protection) or carfentrazone-ethyl (e.g. Spotlight Plus; Berner), or a combination thereof. Weed is preferably removed at a regular basis, for example at weekly or biweekly intervals.

Seedling tubers are preferably harvested when the seedling plants have reached a height of 50-150 cm, preferably of 60-120 cm. With temperatures between 15 and 22° C., and light levels of at least 50 μMol/m2/sec during the light phase of a diurnal pattern, the majority of seedling tubers may be harvested in about 13-20 weeks, such as about 15-18 weeks, after transplanting.

Said harvested seedling tubers may subsequently be planted to produce the commercial crop of potatoes that are then consumed, stored for a period of time, or used as the commercial tuber crop for consumption. Said seedling tubers may be stored for at least one month, such as from autumn to late winter or early spring. Said seedling tubers are preferably stored in a climate-controlled storage room at 2-10° C., such as 4-6° C.

Seedling tubers that are to be planted preferably have a sprouting score of at most 5. Seedling tubers that are to be planted preferably have a square measure of more than 25 mm. Seedling tubers that are to be planted preferably have no visual defects. Seedling tubers are preferably planted with a spacing of at least 15 cm between the seedling tubers, such as 15-50 cm. Seedling tubers are preferably planted in ridges of 10-100 cm, such as 40-75 cm.

Potato plants as defined herein, both seedlings and commercial crops that produce potatoes for consumption, as defined herein produce tubers with a uniform tuber shape, a uniform tuber number per plant, a uniform tuber weight per plant, or a combination thereof. Said potato plant preferably is an essentially diploid potato plant with a coefficient of variation in tuber shape of less than 26 percent. Said essentially diploid potato plant preferably is a diploid F1 hybrid Solanum potato plant. A diploid potato plant of invention preferably produces at least 30 tubers per square meter.

The resulting potato tubers, both seedling tubers and consumption tubers, have a coefficient of variation in tuber weight of less than 26 percent, such as less than 22 percent, or less than 20 percent, a coefficient of variation in tuber shape of less than 26 percent, such as less than 22 percent, or less than 20 percent, or both.

Said potato tubers preferably are essentially homozygous, diploid Solanum potato tubers, or essentially heterozygous, diploid Solanum potato tubers. Said essentially heterozygous, diploid Solanum potato tubers may be obtained by crossing essentially homozygous diploid parent plants as described herein.

### Example 1—Breeding of F1 Hybrid Potato Varieties

In the winter of 2015-2016, crosses between highly homozygous, fertile, and self-compatible Solanum potato inbred parent lines were made manually to produce diploid hybrid seeds. The highly homozygous, fertile, and self-compatible Solanum potato inbred plants were obtained from crosses originating with AGVD1, AGVD2, AGVD3, and AGVD17. A total of 572 hybrids were grown in one location in a first year trial to select the most uniform hybrids. This resulted in a panel of diploid hybrids that was used in the present study. The hybrids described and claimed herein were selected based on yield and yield components to represent the genetic variation among these hybrids and to allow a detailed analysis.

### Example 2—Production of Seed Tubers and Seedling Tubers

Seedling tuber production from true diploid hybrid seeds was performed on a heavy marine clay in Wolphaartsdijk, the Netherlands in 2016. The botanical seeds of the diploid hybrids were extracted from the berries and sown into 104 plug trays (2.5×2.5×3.8 cm) filled with soil (Horticoop) in a greenhouse compartment in April 2016. Germination after two weeks was between 54% and 98%. Six weeks after sowing, the seedlings were mechanically transplanted into the open field into ridges of 75 cm width and a plant distance of 30 cm within the row. The crops were treated as a standard seed tuber crop according to farmers' practices. Irrigation was applied at one and three days after planting to prevent the seedlings from drying out. Pests and diseases were chemically controlled. Both mechanical weeding and hand-weeding were applied twice. After 90 growing days in the field, the haulm was chemically killed. Three weeks after haulm killing the seedling tubers of all diploid hybrids were lifted by a harvesting machine. The seedling tubers were stored in a climate-controlled storage room at 4° C. from October 1st until the end of February.

Seedling tuber production was done in a non-replicated design with a plot size between 90 and 488 plants, depending on hybrid seed availability. Seedlings were transplanted semi-mechanically with an adjusted 4-row cabbage planter with a planting distance of 30 cm between plants. After harvest, seedling tubers were sorted by size and yield was determined for tubers >28 mm. Phytosanitary control on quarantine diseases was routinely carried out by the national inspection service (NAK at Emmeloord, The Netherlands). To assess the quality of the seedling tubers, sprouting was scored in February, at the preparation of the yield trials according to a 1-9 scale, with 9 being completely dormant and 1 being abundantly sprouting, according to the method of Tiemens-Hulscher et al., 2013.

To perform yield trials in which diploid hybrids could be benchmarked against tetraploid commercial cultivars, we first produced seedling tubers from diploid hybrid seeds. This was done to decrease the difference in starting material, as plant performance is largely affected by the starting material. The breeding process of the test hybrids that were used in this trial was done exclusively in the greenhouse, so no selection for yield was applied.

Seedling tubers that were produced were used in the subsequent year for yield trials. The physiological age of seed tubers is one of the main determinants for seed tuber quality and it is affected by many genetic and environmental factors like management of the seed tuber crop, storage conditions, seed tuber size and the state of dormancy. Before the seed tubers were selected for the ware crop trial, sprouting was scored. The results showed that dormancy was broken in all tubers, however there was some variation probably due to different genetic background of the diploid hybrids.

To minimize the experimental error, caused by differences in the physiological conditions of the tubers, seed tubers were stored under optimal conditions and equally sprouting tubers were selected for the yield trial. If present, the largest unexplained error would occur in the diploid hybrids, due to differences in physiological conditions as the tubers of the different hybrids were not stored under optimal conditions for the each hybrid. In addition, some genetic variation within the hybrids may still occur as the parents were not 100% homozygous. However, the within-plot variation of the hybrids was not higher than this variation within commercial cultivars (data not shown). This supports the conclusion that the differences between the hybrids and the commercial cultivars were due to genetic differences.

### Example 3—Yield Trials

In 2017, yield trials were conducted using the seedling tubers as starting material that were produced in 2016. Nineteen commercial tetraploid cultivars were used as controls for the different potato product types. The commercial tetraploid cultivars represent the frying, chip and fresh consumption market: Annabelle, Lady Cristil, Lady Claire, Pirol, Innovator, Lady Rosetta, Agata, Arsenal, Hermes, Brooke, Nicola, Bintje, Markies, Russet Burbank, Fontane, Spunta, Mozart, Agria and Milva. The main difference between these cultivars is dry matter content, shape, and tuber size. The diploid hybrids were not yet differentiated for the different market segments. These control cultivars are commonly grown in the regions where the trials took place. Hence these controls provide a realistic representation of farmers' practice.

Seed tubers of commercial cultivars were classified in class A in the NAK (Nederlandse Algemene Keuringsdienst voor Zaaizaad en Pootgoed van Landbouwgewassen, The Dutch General Inspection Service) classification system. Seed tubers were produced and stored in the conditions optimal for the specific cultivar for a realistic representation of the cultivar's potential. Accordingly, storage conditions were not identical for all cultivars. Diploid hybrids were all stored at 4° C. as the optimal conditions for each specific hybrid had not yet been determined. Seed tubers of equal size and sprout distribution were selected for the trial. To assess the suitability of the seedling tubers for a ware crop trial, sprouting was scored after six months of storage (data not shown). Generally, seed tubers should be planted when dormancy has been broken. Seedling tubers with a sprouting score between 3 and 7 were used in the trial. The sprouting of seed tubers of tetraploid cultivars was comparable to that of the diploid hybrid seedling tubers. The method of Tiemens-Hulscher et al., 2013, incorporated herein by reference, was used to determine the sprouting score.

Certified seed tubers (class A) of the tetraploid cultivars were obtained to serve as control cultivars in the trials. The field trials were conducted at five different sites: Warmeriville, France; Berthem, Belgium; Hilvarenbeek, The Netherlands; Est, The Netherlands, and Giethoorn, The Netherlands. These sites differed in soil type, climate and crop management (Table 1). Two of the sites were typical sandy soils, one a loamy soil, one a light clay, and one a heavy clay soil, covering the full range of physical soil types on which potatoes are grown in NW Europe.

At all sites the trial was laid out within a farmers' field. Crop management was done according to farmers' practice. Typically, the tubers were planted in ridges, spaced at 75 cm, except for the experiment in Warmeriville, where the distance between ridges was 90 cm (Table 1). Spacing between seed or seedling tubers in all locations was 25 cm. In Hilvarenbeek, the farmer had adopted a bed system, which was better adapted to his conditions. Due to the fine sand at this location, ridges would be prone to erosion.

TABLE 1
Location, soil characteristics, crop management system, planting
and haulm killing dates of the five experimental sites.

| | | | | | | | | | Haulm |
|---|---|---|---|---|---|---|---|---|---|
| | Longitude | Latitude | | | | Ridge/Bed | | Planting | killing |
| Site | (E) | (N) | % day | % org C | pH | system | | date | date |
| Warmeriville | 4.219 | 49.345 | 13 | 4.2 | 7.6 | 90 | cm ridge | 19 Apr. 2017 | 29 Aug. 2017 |
| [FR] | | | | | | | | | |
| Hilvarenbeek | 5.086 | 51.483 | 2 | 3.1 | 6.8 | 150 | cm beds | 21 Apr. 2017 | 6 Oct. 2017 |
| [NL] | | | | | | | | | |
| Est [NL] | 5.329 | 51.845 | 33 | 3.7 | 6.1 | 75 | cm ridge | 14 Apr. 2017 | 15 Sep. 2017 |
| Berthem | 4.633 | 50.871 | 13 | 2.2 | 6.4 | 75 | cm ridge | 25 Apr. 2017 | 2 Oct. 2017 |
| [BE] | | | | | | | | | |
| Giethoorn | 6.054 | 52.671 | 1 | 7.4 | 5.4 | 75 | cm ridge | 24 Apr. 2017 | 5 Oct. 2017 |
| [NL] | | | | | | | | | |

During preparation of the field trials, the weight of the seed and seedling tubers for each plot was recorded. Trials were planted mechanically in a randomized complete block design, with three repetitions and 20 plants per plot, divided over two rows. Between each plot two border plants of the cultivar Miss Blush were planted to obtain an equal border effect over all plots. The bi-colored cultivar Miss Blush was chosen to avoid mixture at harvest, as test cultivars and diploid hybrids showed uniform skin color. The whole experiment was bordered by two ridges of Miss Blush at the sides and one full plot of Miss Blush at the beginning and end of each ridge. During the growing season, stem number was counted per plot. After harvest, tuber number and size were measured automatically using a 3D camera (RMA Techniek, 's Heer Arendskerke, Netherlands). Total tuber weight was determined per plot. A subsample of approximately 5 kg was used to determine dry matter content (see below for formulas).

The data from the yield trials were statistically analyzed using R (R Core Team 2017). A component analysis was done on total yield (Formula 1) to determine how these components contribute to total yield in each cultivar. Yield and yield components were adjusted for spatial field trends with Spatial Analysis of Field Trials with Splines (SpATS) (Rodriguez-Alvarez et al. 2018). In this model, differences in plant development due to environmental variation within the field are distinguished from differences in the genetic compositions between the cultivars. In SpATS, variation in the field is analyzed in two directions (rows and columns of the trial) using P-splines, effects of spatial variation are described by a standard mixed model (Rodriguez-Álvarez et al. 2018). By adjusting the data using SpATS, effects of environmental variation within trial fields was reduced, as the focus of this analysis was the genetic differences and the differences between trial fields. To meet the assumptions of normality of the SpATS model, data were log transformed, means and standard errors were back-transformed to linear data for graphical presentation. To examine to which extend the different yield components contribute to total yield, the variation in each yield component was compared with variation in total yield by linear regression. The contribution was defined as the change in yield by changing one unit of the yield component. This was determined by the slope of a linear model, which was fitted through the data of all sites. Genotype by environment interaction was calculated with the AMMI analysis (Additive Main effects and Multiplicative Interaction models), using the Agricolae package in R (Crossa 1990). Results of the AMMI analysis are presented in FIG. 2 and in Table 2. For the AMMI analysis the raw data were used, the rest of the presented data of yield, stems/m2, tubers/stem, and tuber size were adjusted for spatial effects within the field using SpATS. Under water weight was used to determine dry matter content (Formula 3, 4) (Tiemens-Hulscher et al. 2013). Tuber shape was determined using Formula 5.

$$Formula 1 : yield ( gm2 ) = tubersstem \times stems / {m}^{2} \times gram / tuber$$

$$Formula 2 : {y}_{ij} = Block + {row}_{r ( j )} + {col}_{c ( j )} + f ( row , col ) + {g}_{i} + {e}_{ij}$$

$$Formula 3 : Under water weight = — \times h$$

$$3 : h = \frac{5050}{h} \times h$$

$$4 : ( \% ) = h \times 4.90713 \times 10 + 2.054$$

$$5 : h = 1 / \frac{h}{\overline{( \times ( 0.5 \times h ) ) \times ( 0.5 \times h h ) )}}$$

In contrast to regular potato seed tuber production, seedling tubers were produced from diploid hybrid plant seedlings. After seed germination and transplant of plant seedlings to the field, plant seedlings grew rapidly and very few plants died. For most hybrids, the canopy was more or less closed at the end of June. After harvest of the seedling tubers, the number of seedling tubers was assessed and the results are shown in FIG. 1. The present invention provides diploid Solanum potato hybrids that produce 3, 4, 5, 6, 7, 8, 9, and more than 9 seedling tubers per plant which measure. between 25 and 55 mm in size. Although diploid Solanum potato hybrids were not selected for yielding capacity, the present invention provide new cultivars that produce a large number of seedling tubers per plant.

Seedling tubers produced from true hybrid seeds in 2016 were used to conduct multi-location yield trials in 2017. In these trials the performance of diploid hybrids grown from tubers was compared to the performance of tetraploid cultivars in different environments. Diploid hybrids that were used in this experiment were the first test hybrids derived from a hybrid breeding system based on homozygous inbred lines. It is known that the correlation between yield of single greenhouse-grown plants and larger blocs in the field of the same cultivar is low. Therefore, traits for high yield were not considered as selection criteria during the selection of parent lines for these hybrids. Surprisingly, even though yield and tuber uniformity traits were not selected for among greenhouse plants, the hybrids demonstrated excellent yield and uniform tuber traits including shape, flesh color and skin color.

Among the different sites, significant differences (p<0.001) in yield were found (Table 2). The highest yielding site was Hilvarenbeek, with on average 62.5 Mg/ha, while the lowest average yield was measured in Berthem (30.7 Mg/ha). Average yield for the Netherlands and France was 43 Mg/ha, in Belgium 50 Mg/ha (Eurostat), so differences in production between sites were due to local environmental and field management differences.

Fresh tuber yield differed significantly among cultivars (p<0.001). Diploid hybrids yielded on average between 16 and 52 Mg/ha. For the tetraploid cultivars, average yield was between 52 and 101 Mg/ha. Performance of cultivars among the different sites varied. A significant interaction (GxE, p<0.001) in yield between cultivar and site was found (Table 2).

TABLE 2
Analysis of variance table, calculated using the AMMI analysis
(Additive Main effects and Multiplicative Interaction models).

| | | Sum of | | |
|---|---|---|---|---|
| Factor | Df | squares | F value | p value |
| Location | 4 | 160537 | 239.0 | <0.001*** |
| Replicate | 10 | 1679 | 1.8 | 0.05 |
| Genotype | 83 | 448198 | 59.0 | <0.001*** |
| Genotype × | 332 | 104915 | 3.5 | <0.001*** |
| Location (G × E) | | | | |
| Residuals | 789 | 72212 | | |

The genotype x environments interaction of the hybrids and cultivars was analyzed using the AMMI analysis. The AMMI parameters can be used to make a biplot in which the interaction effects of genotype and environment are shown (Gauch and Zobel 1997). In the biplot (FIG. 1) 62.9% of the GxE interaction is explained in the principal component axis PC1. Also the principal component axis PC2 explained a significant part of the G x E interaction (23.6%). The rest of the PC axis were not significant. On average, the yield of diploid hybrids was lower than the yield of tetraploid cultivars. Importantly, however, the hybrids with the highest yields had yields that were similar to the lowest yielding tetraploid cultivars. For these higher yielding hybrids the interaction with the environment was also similar to several tetraploid cultivars (FIG. 1). The large distance between the trial locations in FIG. 1 shows that they differed in environmental effect, except for Warmeriville and Berthem, that were very close to each other.

Dry matter percentage (DM %) was measured in each genotype on all fields separately. A wide genetic variation for DM % was observed, as well as variation in DM % among the different sites. Diploid hybrids showed a broad range of DM %, between 15.8 and 21% (FIG. 3). This was in range with most of the tetraploid cultivars except for three chip cultivars with a DM % of more than 22.5% (Brooke, Pirol and Lady Rosetta). The rest of the chip and frying cultivars had a DM % between 19.2 and 21.3%. A total of 12 of the diploid hybrids also had a DM % within this range. The rest of the hybrids had a DM % comparable with the tetraploid table cultivars that produced a DM % between 19.2 and 15.6. In general, the diploid hybrids had similar DM % to the tetraploid cultivars.

To compare the extent of the environmental effect on genotypes, the coefficient of variation (CV) of DM % over the sites was calculated for each diploid hybrid and tetraploid cultivar (FIG. 3). Variation of DM % between fields in most diploid hybrids was in the same range as the variation of DM % in tetraploid cultivars. The most stable diploid hybrid (Hybrid 1) had a CV of 1.2%, which was similar to the CV of the tetraploid cultivar Innovator. The DM % in Hybrid 1 was 21% and higher than the 19.4% measured in Innovator. Significantly, the dry matter percentage and stability in different environments of hybrids was as good as, and sometimes even better than, in tetraploid cultivars.

Tuber shape is an important trait for cultivars in the chip and the frying industry because tubers need to be suitable for the processing machines (Tiemens-Hulscher et al. 2013). Tuber shape is here expressed by Formula 5, in which the length, width, and height of individual tubers is taken into account. The result is a dimensionless number between 1 and 0, with 1 representing a perfect sphere. Without selection for specific tuber shape in the diploid hybrids during the breeding process, the same range from round to long was found in hybrids as in tetraploid cultivars (FIG. 4). The variation for long tubers for fries represented by frying cultivars like Innovator and Russet Burbank was similar to the variation of several hybrids. Likewise, the variation for round tubers for chips represented by chip cultivars like Hermes and Lady Rosetta was similar to the variation of several hybrids.

The uniformity of the shape is important for mechanical processing. Therefore, the coefficient of variation of the shape data of the individual tubers was calculated for each hybrid and cultivar (FIG. 4). Most diploid hybrids had a higher CV than the tetraploid cultivars. On average the CV for the hybrids was 20% and for the tetraploid cultivars it was 17%. However, the most stable hybrids showed overlap with the least stable tetraploid cultivars. To gain insight into the effects of the different environments on the shape distribution, we show boxplots of two cultivars (FIG. 5). Both Hybrid 9 and Bintje had a stable shape across the different environments. The differences in mean shape and variation between the five locations were smaller for Hybrid 9 and cultivar Bintje.

Potato yield was analyzed as the product of different plant components that can contribute to yield. Individual tuber weight, number of tubers per stem, and number of stems per surface unit were chosen as relevant yield components to compare the growth of diploid hybrids and tetraploid cultivars. These yield components were compared between tuber grown diploid hybrids and commercial tetraploid cultivars that grew in yield trials on five different locations.

Generally, the yield components differed between the tetraploid cultivars and the diploid hybrids. The mass of the individual tubers of diploid hybrids was lower than that of the tetraploid controls (71 g vs. 177 g, on average). The hybrids had on average a higher number of stems (25.6 stems/m2), than the tetraploids (14.9 stems/m2). The number of tubers per stem was higher in the tetraploids compared to the diploids (2.9 and 1.8, respectively). This resulted in approximately equal number of tubers per unit area (45.2 and 43.5 tubers/m2 for diploids and tetraploids, respectively) and all hybrids performed with the number of tubers/m2 within the range of the tetraploid cultivars.

When comparing yield components of individual tetraploid cultivars and diploid hybrids the differences were small. A positive correlation was found between yield and tubers per stem (p<0.001). Indeed, the higher yielding hybrids had a relatively high number of tubers per stem. Hybrid 9, for example, produced 2.3 tubers per stem on average which was nearly as much as Annabelle (2.6) and more than Innovator (1.9). Tuber size also was positively correlated with yield (p<0.001). For those diploid hybrids with larger tubers has higher yields, even though all hybrid tubers were relatively small.

The cultivars with higher yield also had a relatively high number of tubers per m2. For example, Bintje showed a high number of tubers/m2 (60.3) and the highest yield (7.2 kg/m2) across all cultivars, while Hybrid 1 had the lowest yield (1.6 kg/m2) and a low number of tubers m-2 (31.2). In higher yielding hybrids, the number of tubers/m2 was also high, with both Hybrid 9 and tetraploid cultivar Bintje having the largest number of tubers/m2.

TABLE 3
Average values of adjusted yield and adjusted yield components of a selection of representative hybrids
and tetraploid cultivars and adjusted yield, adjusted yield components and effect of yield components
on total yield per cultivar (regression coefficient). Average values of yield components and yield
were calculated using the full dataset of diploid hybrids and tetraploid cultivars.

| | Yield | | | | | | Contribution yield components to yield | | |
|---|---|---|---|---|---|---|---|---|---|
| | Seed(ling) | | | | | | Contribution | Contribution | Contribution |
| | tuber | | Yield components | | | | per stem | tuber size | tuber number |
| Cultivar, | weight | | Tubers/ | Stems/ | Tubers/ | g/ | [g/m2/ | [g/m2/ | [g/m2/ |
| hybrid | (kg/plot) | kg/m2 | m2 | m2 | stem | tuber | (stems/m2)] | (g/tuber)] | (tubers/stem)] |
| Hybrid 1 | 0.72 | 1.6 | 31.2 | 29.9 | 1 | 48 | 84 | 104 | 3161 |
| Hybrid 2 | 0.91 | 2.8 | 31.1 | 17.9 | 1.7 | 89 | −20 | 90 | 1822 |
| Hybrid 3 | 0.71 | 2.9 | 51.9 | 34.1 | 1.5 | 56 | −174 | 145 | 2020 |
| Hybrid 4 | 1 | 3.3 | 36.1 | 19.6 | 1.8 | 89 | 160 | 62 | 2124 |
| Hybrid 5 | 0.82 | 3.8 | 54.7 | 31.3 | 1.7 | 71 | −2 | 119 | 2033 |
| Hybrid 6 | 0.97 | 3.8 | 43.7 | 22.4 | 2 | 91 | 96 | 1 | 426 |
| Hybrid 7 | 0.76 | 3.8 | 52 | 19.8 | 2.6 | 68 | 867 | 179 | 2642 |
| Hybrid 8 | 0.83 | 4.8 | 56 | 25.5 | 2.2 | 85 | 127 | 126 | 2305 |
| Hybrid 9 | 0.73 | 5.2 | 60.6 | 26.4 | 2.3 | 93 | −111 | −8 | 1617 |
| Annabelle | 1.14 | 5.2 | 44.6 | 17.4 | 2.6 | 117 | 165 | 5 | 2123 |
| Innovator | 1.44 | 6.4 | 29.8 | 16 | 1.9 | 222 | 322 | −41 | 3387 |
| Hermes | 0.45 | 6.9 | 43.1 | 11.6 | 3.7 | 159 | 956 | −101 | 2153 |
| Bintje | 0.51 | 7.2 | 60.3 | 16 | 3.8 | 125 | 257 | −42 | 1164 |
| SITE | | | | | | | | | |
| Berthem | — | 3 | 38.6 | 27.6 | 1.4 | 93.4 | — | — | — |
| Warmeriville | — | 3.1 | 37.4 | 20.9 | 1.8 | 99 | — | — | — |
| Est | — | 3.3 | 36.6 | 18.5 | 2 | 98 | — | — | — |
| Giethoorn | — | 5.3 | 60.8 | 25.3 | 2.4 | 90 | — | — | — |
| Hilvarenbeek | — | 5.5 | 63.7 | 23.7 | 2.7 | 96 | — | — | — |

Variation in yield was found across sites as well as between cultivars. The variation in yield was due to the difference in responses of individual genotypes to the environments. Although there are trade-offs and interdependencies between yield components, they explain differences in yield between cultivars and environments.

The number of tubers per stem showed the largest variation across sites (CV=24.8%). Between Berthem and Hilvarenbeek, the difference in number of tubers per stem was almost a factor two. For stems/m2 the variation was much lower (CV=15.3%) and the weight of the tubers was rather stable (CV=3.7%). In conclusion, the yield component that was affected most by the environment was the number of tubers per stem.

To compare the extent to which different yield components were affected by the environment between cultivars, the contribution of individual yield components to total yield over the different sites was determined by calculating the slope between total yield and yield component across sites (Table 3). It estimated the yield gain for a single yield component for each cultivar, with a positive value indicating a contribution to higher yield when the specific yield component increases, and a negative value indicating a loss in total yield when increasing a specific yield component.

To compare the relative effects of yield components on yield between the tetraploid cultivars and the diploid hybrids, correlations between yield components and yield were calculated. Every pair of average yield and yield component value of each location was used in the regression analysis. Data are shown in the last three columns of Table 3. It compared the difference in contribution to yield of yield components. Number of tubers per stem explained 80% of the variation in yield in tetraploids and 90% in the diploid hybrids. The relative effect of number of stems per m2 on yield was much smaller: 18% in tetraploid cultivars and 5% in hybrids. Tuber size affected yield least in tetraploids and hybrids (2% and 5%, respectively). On average, hybrids and tetraploid commercial cultivars shared the most important contributing factor to yield.

A higher number of tubers per stem contributed positively to total yield in all cultivars (Table 3). For diploid hybrids in the selection of Table 3, the contribution was between 426 and 3161 g yield increase per m2 for each extra tuber per stem. In the tetraploids this was between 1164 and 3387 g/m2 yield increase/(tuber/stems). More stems per m2 led in some hybrids to a decrease in total yield while in others total yield increased with more stems per m2 (contribution between −174 and 867 g/m2 yield increase/(stems/m2) in Table 3). In the tetraploid cultivars higher stem numbers were positively correlated with yield with contributions between 165 and 956 yield g/m2 yield increase/(stems/m2). There was a large variation in the contribution of larger tubers to total yield between diploid hybrids as well as in tetraploid cultivars, with values between −8 and 175 g/m2 yield increase/(g/tuber) for hybrids and −101 and 5 g/m2 yield increase/(g/tuber) for tetraploid cultivars. Overall, the range of yield contribution by the different yield components was overlapped between diploid hybrids and tetraploid cultivars.

Variation was found between the different cultivars in total yield as well as in the contribution of the separate yield components to yield. To examine whether there is an optimal value for each yield component for high yield, the gain in yield with an increase of the yield component was compared with the average value of that yield component for each cultivar. Although yield gain and increase in yield component are not completely independent, it provides insight into whether there are optimal yield components values.

Genotypic variation was found for the contribution of yield components to total yield. The effect of differences in yield components between genotypes on the correlation between yield component and yield was examined (FIGS. 6, 7, 8). Most genotypes showed a positive correlation between yield and tubers/stem when the average number of tubers/stem was between 1.0 and 3.8, so for this dataset more tubers per stem contributed positively to total yield in the whole range.

In contrast to the number of tubers/stems, larger tubers also affected yield negatively in some cultivars (FIG. 4). A negative relation was found between contribution of larger tubers to total yield and average tuber size of a genotype (FIG. 5). Until a tuber size of about 90 g the production of larger tubers resulted in a higher total yield. When a genotype already produced large tubers on average, larger tubers decreased total yield. So with increasing average tuber size of a genotype contribution of even larger tubers to total yield decreased.

TABLE 4
Sprouting Score of Seedling Tubers.

| | Number of | | |
|---|---|---|---|
| | seedling tubers | | Sprouting score after six |
| Diploid | (size 28-55 mm) | Yield | months of storage |
| hybrid | m-2 | (kg/m2) | (February, 2017) |
| Hybrid 1 | 15 | 0.6 | 3 |
| Hybrid 2 | 12 | 0.9 | 5 |
| Hybrid 3 | 31 | 1.4 | 3 |
| Hybrid 4 | 25 | 1.5 | 5 |
| Hybrid 5 | 19 | 0.9 | 5 |
| Hybrid 6 | 15 | 0.8 | 2 |
| Hybrid 7 | 30 | 1.4 | 5 |
| Hybrid 8 | 26 | 1.6 | 5 |
| Hybrid 9 | 27 | 1.3 | 3 |

In addition to the number of seedling tubers produced per m-2, the ability to form sprouts was also evaluated. As shown in Table 4, following six months of storage, all hybrids produced seedling tubers with a sprouting score greater than 5. The method of scoring sprouting described in Tiemens-Hulscher M, Delleman J, Eising J, Lammerts van Bueren E T (2013) Potato Breeding. Drukkerij De Swart, Den Haag, incorporated herein by reference, was used to determine the sprouting score. The scoring scale is from 1 to 9 with 1 representing a high level of sprouting and 9 representing no sprouting.

### Example 4—Effect of Temperature on Germination

In 2015 a germination trial was performed using five different diploid essentially homozygous inbreeding lines. The trial was done on a temperature gradient table with a temperature range from 10 to 30° C. Germination was scored at 4, 7, 11 and 14 days after sowing. In all breeding lines, germination decreased substantially at a temperature higher than 20° C. (FIG. 9), reducing to about 50% at 25° C. In later trials the same trend in temperature sensitivity was shown for F1 hybrids, although these hybrids seem to be somewhat more heat resistant, when compared to the diploid essentially homozygous inbreeding lines (see FIG. 10).

### Example 5—Effect of Light on Germination

A seedling trial was performed using 10 different diploid hybrids and four different light intensities (50, 100, 150 and 300 mol/m²/s). The trial was laid out in a split plot design with light intensity as main plot and hybrid as subplot, with three replicates and ten plants per plot. Plant length and fresh weight was measured for each seedling. With increasing light intensity, seedlings produced a higher fresh weight (FIG. 11). Plant length on the other hand was highest under the lowest light intensity, and plants were shortest under the high light intensity (data not shown). So with increasing light intensity seedlings produced more biomass over a shorter length, leading to stronger plants. FIG. 12 gives an image of seedlings grown under the different light intensities. However, despite the slow growth, the seedlings do grow at a light intensity of 50 mol/m²/s. So it is possible to produce hybrid seedlings at al light intensity of 50 mol/m²/s or higher, with increasing plant quality when light intensity increases.

### Example 6—Transplanting to the Field

Seedlings are preferably transplanted at a temperature higher than 0 degrees. This was shown in two different field trials. In the first trial, seedlings of four different hybrids that were planted in April died due to frost, while seedlings of the same hybrid survived in two later transplant moments. In a second trial, seedlings of two different hybrids and two different ages were transplanted in March, April and May. The seedlings in March had temperatures below 0 degrees and died, the seedlings from the other transplant moments survived.

### Example 7—Seed Tuber Storage

Two different hybrids (SOLHY0001 and SOLHY0006) were grown from true seeds as well as from seedling tubers in a seed tuber production field in Oud-Gastel (NL). Plantlets grown from true seeds were planted and grown as described by Stockem et al. (2020). Seedling tubers were planted like in conventional seed tuber production. Plants from true seeds and seedling tubers were grown in one field and treated the same way. After harvest (beginning of November) the tubers were stored under three different conditions (Table 5). At the start of harvest no sprouting was detected. Sprouting was scored five times in the period between harvest and planting.

TABLE 5
Different storage conditions and temperatures.

| Location | Type of storage | Degrees | |
|---|---|---|---|
| Slijk-Ewijk | All purpose | 8-12° | C. |
| Giethoorn | Fries, Fontane | 6-7° | C. |
| Est | Fries, Fontane | 7-8° | C. |

Sprouting over time for seed tubers grown from true seeds and seedling tubers is shown in FIGS. 14 and 15, respectively. Seed tubers grown from true seed started sprouting early (in February) when stored at a temperature higher than 7° C. Seed tubers grown with seedling tubers as starting material sprouted in February as well when stored at 7-8° C. When stored at 8-12° C. they started sprouting even earlier, in January.

These results lead to the conclusion that hybrid seed tubers grown from true seed as well as seedling tubers should be stored at a temperature lower than 7° C. to ensure a good quality of seed tubers at the moment of planting.

### Example 8—Effect of Light on Germination

Seedling growth can be steered by increasing fertilization when the rest of the climate conditions are good for seedling growth. In 2020 a seedling trial was performed in which fertilization was increased up to 3 dS/m. For this, seedlings of two different hybrids were grown under fertilizer (Peters Professional 20-20-20+TE) with an electric conductivity (EC)=0.9 ds m-1 (control), 1.5 dS m-1, 2.0 dS m-1 and 3.0 dS m-1. Fertilizer was applied three times per week, starting 15 days after sowing. The trial was set up in a completely randomized block design with four replicates and eight plants per replicate. At harvest, fresh and dry weight, leaf area, number of leaves, stem length and stem firmness were measured. Growing parameters like leaf area and biomass production was significantly higher compared to the standard of approximately 0.9 dS/m (FIG. 13, Table 6).

TABLE 6
Average values and significance of different plant traits
measured in hybrid seedlings six weeks after sowing.

| | Freash | Cumulative | Leaf area | | Number of | Stem length | | Stem firmness | |
|---|---|---|---|---|---|---|---|---|---|
| Treatment | weight (g) | dry weight (g) | (cm2) | | leaves | (cm) | | (g/cm) | |
| EXP 1 | | | | | | | | | |
| SOLHY004 | | | | | | | | | |
| Control1 | 1.1 a | 2.2 a | 22.8 | a | 5.8 a | 7.2 | a | 0.05 | ab |
| Control 2 | 1.1 a | 2.3 a | 24.3 | a | 5.9 a | 6.6 | a | 0.05 | a |
| EC1.5 | 2.4 b | 4.0 b | 49.9 | b | 6.6 b | 12.3 | b | 0.08 | ab |
| EC2.0 | 3.5 c | 5.1 c | 77.3 | c | 7.3 c | 16.1 | c | 0.09 | b |
| EC3.0 | 4.4 d | 5.6 c | 81.5 | c | 7.6 c | 18.1 | d | 0.11 | b |
| EXP 1 | | | | | | | | | |
| SOLHY007 | | | | | | | | | |
| Control1 | 1.2 a | 2.1 a | 27.4 | a | 6.2 a | 5.3 | a | 0.06 | a |
| Control 2 | 1.1 a | 2.2 a | 27.4 | a | 6.1 a | 5.2 | a | 0.06 | b |
| EC1.5 | 2.6 b | 3.8 b | 61.1 | b | 7.2 b | 11.5 | b | 0.08 | ab |
| EC2.0 | 3.9 c | 5.0 c | 88.4 | c | 7.7 c | 16.2 | c | 0.09 | b |
| EC3.0 | 5.0 d | 5.8 d | 112.5 | d | 8.1 d | 17.8 | d | 0.11 | c |

What is claimed is:

1. A method of producing uniform potato tubers comprising the steps of:
(a) sowing Solanum potato seed in a receptacle;
(b) growing said seed into seedlings for 3-8 weeks under protected growth conditions, at 15-25° C. and >30% humidity, while supplying light levels of at least 50 μMol/m2/sec during the light phase of a diurnal pattern after germination;
(c) mechanically transplanting the seedlings and growing the seedlings at a temperature of more than 0° C., while maintaining a matric potential pF at between 2 and 4.2 during at least the first three days, and supplying light levels of at least 50 μMol/m2/sec during the light phase of a diurnal pattern;
(d) harvesting the uniform seedling tubers when the plants have reached a height of 60-120 cm; and
(e) optionally, planting the uniform seedling tubers in a subsequent crop cycle to produce uniform potato tubers.

2. The method of claim 1, wherein the uniform potato tubers are characterized by a uniform tuber shape, a uniform tuber number per plant, a uniform tuber weight per plant, or a combination thereof.

3. The method of claim 1, wherein the potato tubers are essentially homozygous, diploid Solanum potato tubers.

4. The method of claim 1, wherein the potato tubers are essentially heterozygous, diploid Solanum potato tubers.

5. The method of claim 4, wherein the essentially heterozygous, diploid Solanum potato tubers are obtained by crossing essentially homozygous diploid parent lines.

6. The method of claim 1, wherein the seeds are grown for 3-8 weeks in a greenhouse.

7. The method of claim 1, wherein the seeds are sown with a spacing of at least 4 cm between the seeds.

8. The method of claim 1, wherein the seedling tubers are planted with a spacing of at least 15 cm between the seedling tubers, such as 15-5-cm.

9. The method of claim 1, wherein seedlings with more than 4 leaves, with a height of more than 3 cm above soil surface, or both, are mechanically transplanted.

10. The method of claim 1, wherein seedling tubers are planted that have a square measure of more than 25 mm and/or have no visual defects.

11. The method of claim 1, wherein the seedling tubers that are planted have a sprouting score of at most 5.

12. The method of claim 1, wherein the seed is grown at 15-20° C. and >80% humidity until sprouting, and at 15-25° C. and >30% humidity thereafter.

13. The method of claim 1, wherein the potato seed was obtained by a method comprising the steps of:
(a) crossing a plant of a first diploid, self-compatible, fertile, highly homozygous potato line with a plant of a second diploid, self-compatible, fertile, highly homozygous potato line, whereby the second potato line differs in at least 20 homozygous loci as determined by molecular marker analysis, when compared to said first potato line; and
(b) collecting seeds produced from said cross.

14. The method according to claim 13, wherein the trait of self-compatibility is controlled by a Sli gene.

15. The method of claim 1, wherein the harvested seedling tubers were stored for at least one month.

16. The method of claim 16, wherein storage for at least one month was performed at a temperature of 2-10° C.

17. A collection of essentially diploid potato tubers obtained from a single plant that, when planted, produce potato plants with a coefficient of variation in tuber shape of less than 26 percent.

18. The collection of claim 17, wherein the essentially diploid potato tubers are essentially diploid potato seedling tubers.

19. An essentially diploid potato plant with a coefficient of variation in tuber shape of less than 26 percent.

20. The diploid potato plant of claim 19, wherein the essentially diploid potato plant is a diploid F1 hybrid Solanum potato plant.

21. The diploid potato plant of claim 19, wherein the plant produces at least 30 tubers per square meter.
